(19) United States
(12) Patent Application Publication
(10) Pub. No.: US 2011/0129547 A1
(43) Pub. Date: Jun. 2, 2011

(54) PREPARATION FOR CARE AND PROTECTION OF SKIN WITH CHILDREN AND ADULTS, METHOD FOR OBTAINING THEREOF

(76) Inventors: Vojislav Pavlovic, Beograd (RS); Ljubica Pavlovic, Beograd (RS)

(21) Appl. No.: 12/994,878

(22) PCT Filed: Jun. 12, 2008
(86) PCT No.: PCT/RS2008/000018

§ 371 Date: Jan. 31, 2011

(30) Foreign Application Priority Data
May 30, 2008 (RS) P-2008/0245

(51) Int. Cl. A61K 33/30; A61P 17/16; A61P 17/00; A61P 31/02

(52) U.S. Cl. 424/642

(57) ABSTRACT

Preparation for care and protection of skin with children and adults, i.e. method for obtaining thereof consists in that the zinc oxide and powder resolve in paraffin oil during 1-2 minutes up to the complete homogenization, so that these substances can be homogenized and prepared for combining merging with other substances. In the following step the water-free lano-line and white Vaseline are melted and dissolved at the same temperature of about 65% and poured into duplicate unit. Moreover in such a way melted lanoline and Vaseline, paraffin oil is added, mixture of zinc oxide and powder previously dissolved in paraffin oil and citric acid, hence the cooled mass is added. Bisabolol and bitter almond water, with constant mixing up the complete homogenization of mass, i.e. obtaining the consistency of ointment. Preparation obtained by this procedure is cooled and a slight yellowish ointment with fine almond fragrance is obtained.

### TECHNICAL FIELD OF THE INVENTION

Preparation for care and protection of skin with children and adults, method for obtaining thereof, belongs to the field of pharmacy in general and more narrowly to the preparations for cosmetic purpose. More precisely, the invention refers to the preparations characteristic for its special physical form of ointments, i.e. perfumed ointments. According to the International Patent Classification, the invention belongs to the class A 61 K 33/30, and A61P 17/00, A 61K 33/30, 33/22.

### TECHNICAL PROBLEM

Technical problem solved with herewith explained invention includes the following: how to define the ratio of zinc oxide, powder, bisabolol, lanolin, paraffin oil, white Vaseline, bitter almond water, citric acid and some other ingredients, i.e. how to define the method wherein the use of the mentioned ingredients will result with obtaining the preparation for care and protection of particularly sensitive child skin, but also skin of adults, while not decreasing the effect of the main ingredients therewith.

### STATE-OF-ART

There is a range of preparations for care of child and adult skin in both domestic and foreign markets. Such preparations are of worldwide large producers, such as the German producer Nivea”, and some domestic producers (commercial titles of those products are Pavlogal and etc.), as well.

However, besides careful research of the composition of those preparations, the applicant of this invention did not find the preparation which uses all the ingredients used in the invention in the subject, and therewith those preparations cover a very narrow field of application, thus some of them by long term use can have a cumulative effect, as well as allergy and other manifestations (rash, diarhea, etc.).

Domestic patent registered under number YU 48679, the applicant of which is at the same time is the applicant in this application, refers to the Procedure for obtaining a preparation for healing, care and protection of child and adult skin”. The mentioned preparation is in wide use not only in our country but also in many other countries and it showed an extraordinary results in care, prevention an d protection of child and adult skin, particularly against certain reactions on the skin, such as sore spots, scabs, burns, rash, then minor wouds (improves circulation). Moreover, the preparation is used even with the phase of bulla burn disorders where no blisters are broken, and also with the other phase of epithelization, lean face, lean skin and etc.

As a difference from the mentioned preparations, the commercial title of which is “Original Pavlovic Ointment” (Originalna Pavloviceva mast) now field invention contains no boric acid, which is replaced with bisabolol and citric acid, the purpose of which is no more healing and that due to the change in the ingredients, and their percentage in the ointment, the changes reflect to the change not only in the composition, but with regard to the method for obtaining the preparation, that will be further explained in the continuation of the description of here applied invention.

In the state of art, there are technical solutions from the following patent specifications:
1. U.S. Pat. No. 4,381,765, dated on May 10,1983, the applicant of which is Moeller Hihrch et al;
2. U.S. Pat. No. 5,951,993, dated Sep. 14, 1999, the applicant of which is Asmus Robert et. A1;
3. U.S. Pat. No. 143 159, dated Mar. 6, 1979, the applicant of which is Moeller Hihrch et al.

None of the mentioned specifications is opposed to the patentibility of herein filed invention, because of sigificantly different ingredients and significantly different method for obtaining the preparation.

Due to the use of already mentioned ingredients, and the replacement of boric acid with bisabolol and citric acid, the obtained preparation is absolutely untoxic, has no undesired effects, has no contraindications, has no cumulative effect and has no allergy reactions.

### Exposition of the Subject of Invention

This invention solves, according to this invention, the set out technical problem in the corresponding way by obtaining the preparation based on completely natural, untoxic, high quality ingredients explained in the application for the gant of this invention.

The subject of the invention consists in the use of the following ingredients (mass units %, in the continuation of the text mass percents (%)):
pharmacologically active substances (that have mild antiseptic, astringent, emollient and adsorbent effect):
   zinc oxide (Zinci oxydum), from 0.585 to 0.6 mass %;
   talcum powder (Talcum), from 0.585 to 0.6 mass %;
   bisabolol, from 0.29 to 0.310 mass %;
auxiliary substances (have a lubricative, emollient and corrective effect):
   citric acid (Cidric acid), od 0.160 do 0.170 mass %;
   bitter almond water (Aqua amygdalea amarea), from 11.80 to 12.00 mass;%;
   paraffin oil-liquid paraffin (Paraffinum liquidum), from 15.70 to 15.95 mass %;
   waterless lanolin (Cera lanae), from 33.85 to 34.25 mas %;
   white vaseline (Vaselineum album-Petrolatum), from 34.00 to 36.50 mass %, and
   destilled water (Aquae purificta ad) up to 100 mass %.
      to obtain a product with required pharmacological and dynamic properties, so as to have both protective and preventive effect. Hence, the preparation has a slight ceratolitic and nutritive effect, the preparation obtained from this original process is successfully used particularly with children, but with adult, as well,

Substantial difference with here filed application for the preparation is in the use of boric acid powder, being replaced with bisabolol, and in that citric acid (Citric acid) is used as a new ingredient.

Both new ingredients will be briefly explained below:

Bisabolol is a natural preparation extracted from chamomile plant (Chamimile Matricaria recutita), being its active ingredient. Bisabolol has anti-inflammable and antiphlogostic effect. Bisabolol is a colorless fragrance free liquid, dissolved in ethanol, isopropranoi, aliphatic (fatty alcohols) and cosmetic oils, and it is suitable for applying into all pharmaceutical forms for external use. Before recent experiences, bisabolol is used for skin care, in shaving preparations, sunscreen preparations and after sunbathing creams or lotions, for oral hygiene, for hair care, and that in concentration from 0.1-1.0%.

Citric acid (Citric acid) is a white, crystal powder, i.e. colorless crystals or grains, easy water-soluble, alcohol-soluble, but weak ether-soluble substance. It is an apyrogenic substance, meltable at 153° C. Citric acid is used for obtaining solvents for dialysis, oral use, as an antioxidant in cosmetic preparations, and as a means to improve pH value of the preparation.

Preparation for care and protection of child and adult skin, i.e. method for obtaining thereof according to this invention, will be explained with an example of its accomplishment: for the quantity of 100 g of preparation—ointment.

### EXAMPLE 1

Preparation for care and protection of especially child, but also of adults skin, comprising bisabolol and citric acid, is obtained according to the method that includes the following steps:
1. the ingredients used for obtaining the preparation are previously measured at a chemical (pharmaceutical) balance, where for obtaining 100 grams of the ointment the following ingredients are necessary:
   4. 0,595 g. zinc oxide,
   5. 0.595 g. talcum powder,
   6. 0.300 g. bisabolol,
   7. 0.166 g. citric acid,
   8. 15.83 g. paraffin oil-liquid paraffin,
   9. 34.16 g. waterless lanolin,
   10. 35.00 g. white Vaseline,
   11. 11.90 g. bitter almond water and
   12. 1.454 g. distilled water.
after measuring, zinc oxide powder and talcum powder, are dissolved in ¼ of the total quantity of paraffin oil, during 1-2 minutes, so that these substances are homogenized and prepared for putting together with other substances;
in the following step the waterless lanolin and white Vaseline are melted at the temperature of about 65% and poured in a duplicate unit;
into in such a way melted lanolin and Vaseline, the remaining quantity of paraffin oil is added, zinc oxide and talcum powder compound previously dissolved in paraffin oil and citric acid, and then mixed in water bath until the temperature of the obtained mass drops at about 40° C.;
thereupon, into the cooled mass, bisabolol and almond water are added, with constant mixing until the total homogenization, i.e. obtaining the consistency of the ointment.

The obtained preparation is a light yellowish, easy spreadable ointment with almond fragrance.

For this way of obtaining the preparation, depending on the quantity, blender i.e. kitchen mixer is used.

Conditions for keeping in such way obtained preparation, are dry, cool and dark pace, at the temperature of about +25° C.

Besides here represented main version, it is possible to determine new variations with insisting on particular pharmacological properties.

Depending on the intended purpose, preparation is packed into the appropriate packing (plastic or glass small jars or tubes and alike).

The obtained preparation is used with spreading over the skin and gentle massaging, several times a day, for care and protection, primarily for child skin and to prevent contact with irritating substances from urine, feces and perspiration. At the same time, therewith prevented and reduced is a risk for development of sore spots, erytheme, excoriation, being the grounds for infections. Besides the above mentioned, and with application of here filed invented preparation, child skin is treated for health, vital and it restores the required degree of fat and humidity.

The preparation is also used with light irritation disorders on the child skin and adult skin as well (erytheme, scabies, redness), and used for care and protection of adult skin (dry skin, striae, upon sunbathing).

1. Method for obtaining of the preparation for care and protection of skin with children and adults is characterized in, that the zinc oxide powder, in the quantity from 0.585 to 0.6 mass % and talcum powder -soapstone, in the quantity from 0.585 to 0.6 mass %, are dissolved in % total quantity of paraffin oil from 15.70 to 15.95 mass %, during 1-2 minutes up to the final homogenization, that the waterless lanolin in the quantity from 33.85 to 34.25 mass % and white Vaseline in the quantity from 34.0 to 36.50 mass % are dissolved at the temperature of about 65° C. and poured into a duplicate unit, that into in such a way dissolved lanolin and Vaseline, is added the remaining quantity of paraffin oil, mixture of zinx oxide and talcum powder (soapstone) previously dissolved in paraffin oil and citric acid, in the quantity from 0.160 to 0.170 mass %, and mixed in water bath until the temperature of the obtained mass comes down to 40° C., and thereupon in the cooled mass bisabolol is obtained, in the quantity from 0.29 to 0.310 mass % and bitter almond water in the quantity from 11.80 to 12.00 mass %, with constant mixing until the complete homogenization of the mass, i.e. obtaining the consistency of the ointment,

2. Preparation for care and protection of child and adult skin, is characterized in, that it is obtained from the method according to the claim 1.
